(19) United States
(12) Patent Application Publication
(10) Pub. No.: US 2008/0227688 A1
(43) Pub. Date: Sep. 18, 2008

(54) Inhibition of viral replication

(76) Inventors: Randal N. Johnston, Calgary (CA); Manbok Kim, Gainesville, FL (US); Morley D. Hollenberg, Calgary (CA); Kristina K. Hansen, Calgary (CA)

Correspondence Address: FULBRIGHT & JAWORSKI L.L.P., 600 CONGRESS AVE., SUITE 2400, AUSTIN, TX, 78701, US

(21) Appl. No.: 12/075,679

(22) Filed: Mar. 13, 2008

Related U.S. Application Data
(60) Provisional application No. 60/906,706, filed on Mar. 13, 2007.

(51) Int. Cl. A61K 38/00; C12N 7/00; A01N 1/02

(52) U.S. Cl. 514/2; 435/235.1; 435/1.1

(57) ABSTRACT

The present invention provides inhibitors of viral replication, and methods related thereto. In general, such compounds can be classified as peptidyl fluoromethylketones (PFMKs). The PFMK compounds may be used to partially or completely inhibit viral infection. In certain embodiments, Z-FA-FMK may be used to inhibit replication of a reovirus, such as a wild-type or attenuated reovirus, or a leporipoxvirus, such as myxoma virus. These compounds may be useful for controlling viral infectivity in vivo and/or in vitro.

This application claims priority to U.S. provisional patent application Ser. No. 60/906,706, filed on Mar. 13, 2007, the entire contents of which are incorporated herein by reference.

This work was supported by grant no. 73-0520 from the Canadian Institutes of Health Research.

### BACKGROUND OF THE INVENTION

1. Field of the Invention

The present invention relates generally to the field of virology. More particularly, the present invention involves the use of peptidyl fluoromethylketones (PFMKs) for the inhibition of viral replication. In certain embodiments, such inhibition may be useful in treating viral-based conditions. For example, in certain embodiments, the PFMKs may inhibit poxviruses. The PFMKs may be, in certain embodiments, cathepsin B inhibitors or cysteine protease inhibitors. For example, PFMKs that are cathepsin B inhibitors may be used to control reoviral activity in immunocompromised patients undergoing reoviral-based anti-cancer therapy.

2. Description of Related Art

Viruses are potent infectious pathogenic agents that cause important functional alterations of the invaded cells, often resulting in cellular death. Viruses replicate by infecting host cells, thereby giving rise to multiple progeny. It is generally acknowledged that cell injury in viral diseases includes not only direct damage inflicted by the proliferation of viruses, but also various immunologic reactions elicited by infection with viruses. The consequences of a viral disease depend upon several viral and host factors such as the quantity of infecting viral particles, the speed of viral multiplication and spread, the impact on cell functions, the host's secondary responses to the cellular injury, and both the immunologic and the non-specific defenses of the host. In general, the effects of a viral disease include asymptomatic infections, both acute and chronic, clinical diseases and induction of various types of cancers.

Examples of human diseases and conditions caused by viruses include acquired immune deficiency syndrome (AIDS), the flu, chickenpox, Ebola, avian flu, severe acute respiratory syndrome (SARS), smallpox and cold sores. Viral infections can be treated in a variety of ways, depending on the virus and the health of the infected subject. One method of controlling viral infection involves curtailing viral replication. Some examples of viruses, their related diseases and treatments are detailed below.

Rotaviruses, which are members of the Reoviridae family, are the leading cause of severe, life-threatening viral gastroenteritis in infants and animals (Kapikian et al., 1996) and are associated with sporadic outbreaks of diarrhea in elderly (Halvorsrud, 1980) and immunocompromised patients (Holzel et al., 1980). Indeed, rotaviruses are the most common cause of severe diarrhea in children. Moreover, the outcome of infection is age-related: although rotaviruses may infect individuals and animals of all ages, symptomatic infection (i.e., diarrhea) generally occurs in the young (6 months to 2 years in children, and up to 14 days in mice), and the elderly. RotaTeq® (Merck) is presently the only FDA-approved vaccine against rotavirus. Another vaccine, Rotarix® (GlaxoSmithKline), is approved in many countries, but not the United States. Treatment regimens are limited, and include oral rehydration therapy and administration of intravenous fluids. As such, a need exists to develop additional, alternative means of treating rotavirus-related conditions.

Another member of the Reoviridae family is the REO (Respiratory and Enteric Orphan) virus. Reovirus is a ubiquitous, non-enveloped virus containing 10 segments of double stranded RNA as its genome, with human infections that are generally mild, restricted to the upper respiratory and gastrointestinal tracts and often asymptomatic (Tyler and Fields, 1996). Importantly, reovirus has been recognized for many years as displaying striking cytocidal activity when it infects certain types of transformed cells (Duncan and Stanish, 1978; Hashiro et al., 1977). The underlying basis for reoviral oncolytic activity remained unknown until it was shown that transformed cells containing oncogenic Ras-signalling pathways were preferentially susceptible to reovirus infection (type 3 Dearing strain) in vitro and in vivo (Coffey et al., 1998; Kim et al., 2007; Norman and Lee, 2005; Strong et al., 1998). As Ras gene mutations are frequently observed in various types of human cancers (Duursma and Agami, 2003), these findings have led to the current use of reovirus in clinical trials (Norman and Lee, 2005). However, in immune compromised hosts such as newborn and SCID (severe combined immunodeficiency) animals, the wild-type reovirus causes significant viral pathogenesis especially in neural tissue and cardiac muscle tissue (Baty and Sherry, 1993; Loken et al., 2004; Sabin, 1959; Weiner et al., 1977). In some cases, even in immune-competent hosts including humans, the wild-type reovirus has been associated with viral pathogenesis (Hirasawa et al., 2003; Terheggen et al., 2003). Therefore, especially in immune-compromised or very young hosts, the wild-type reovirus does not act always in a benign manner. This wayward activity may prove harmful in certain cancer patients treated with extensive radio/chemotherapy as they can be subject to immunosuppression. Thus, there is a clear need to modulate viral replication pharmacologically in order to turn off undesired viral replication during reovirus anti-cancer therapy.

Smallpox, a devastating infectious disease dreaded throughout much of recorded history, is caused by the variola virus, a member of the Poxyiridae family. In the 20th century alone, smallpox deaths worldwide numbered in the millions. In 1980, after an intensive program of immunization with vaccinia virus, a related but relatively nonpathogenic virus, the World Health Organization (WHO) declared the disease eradicated. By 1983, all known stocks of variola virus were in two WHO collaborating centers: the U.S. Centers for Disease Control and Prevention (CDC) in Atlanta and (after a transfer in 1994) the Russian State Research Center of Virology and Biotechnology (the Vektor Institute) in Novosibirsk. The WHO Committee on Orthopoxvirus Infections voted on several occasions to recommend destruction of the stocks, but each time the decision was deferred to permit more research on live variola virus. A 1999 National Academies report summarized and assessed scientific needs for live variola virus (Institute of Medicine (1999) Assessment of Future Scientific Needs for Variola Virus, ed. Briere, R. (Natl. Acad. Press, Washington, D.C.)). The concern that undeclared stocks of variola virus might exist and that they might be used as a bioterrorist weapon (Lane, et al., 2001) was heightened in late 2001 by the deliberate release of Bacillus anthracis, the agent of anthrax, in the weeks after the Sep. 11, 2001 attacks. That concern prompted a voluntary national preparedness effort to vaccinate healthcare workers, first responders, and members of the military against smallpox. However, given the substantial side effects, the risks associated with the smallpox vaccine, and the absence of information about an imminent bioterrorist attack, vaccination was not accepted by all members of those groups, nor was it recommended for the general public by the government (White House (Dec. 13, 2002) Protecting Americans: Smallpox Vaccination Program, press release, located on the world wide web at whitehouse.gov/news/eleases/2002/12/20021213-1.html). Whatever the likelihood of covertly held variola virus stocks, an intentional release of the virus would pose a serious health threat and would probably provoke a global health crisis. The lethality of the disease (up to 40%) and its ease of transmissibility place variola virus at the top of CDC's list of high-threat (Category A) agents. For that reason, the federal government rapidly increased its support of research related to the discovery of antiviral drugs against smallpox.

### SUMMARY OF THE INVENTION

Thus, in accordance with the present invention, there is provided compounds that modulate viral replication, and methods related thereto. In general, such compounds can be classified as peptidyl fluoromethylketones (PFMKs). Non-limiting examples of PFMKs include N-benzyloxycarbonyl-Phe-Ala-fluoromethylketone (Z-FA-FMK), N-Morpholineurea-Phe-homoPhe-fluoromethylketone (Mu-F-hF-FMK) and N-benzyloxycarbonyl-Val-Ala-Asp-fluoromethylketone (Z-VAD-FMK). Other PFMKs are described herein and are known to those of skill in the art. The modulation may entail partial or complete blockage of viral infection, in vitro and/or in vivo. For example, the present invention encompasses the use of N-benzyloxycarbonyl-Phe-Ala-fluoromethylketone (Z-FA-FMK), a cathepsin B inhibitor, to completely block reovirus infection in vitro and in vivo. Compounds such as Z-FA-FMK may be used to provide pharmacologic control of viral replication.

Accordingly, in certain embodiments, the present invention contemplates a method of inhibiting viral replication in a cell, comprising contacting the cell with an effective amount of a peptidyl fluoromethylketone (PFMK). In certain embodiments, the virus is a Reoviridae virus or a Poxyiridae virus. The PFMK may be a cathepsin inhibitor or a cysteine protease inhibitor, in certain embodiments. Cathpesin inhibition assays are well-known in the art, and at least one is described below in detail. Cysteine protease inhibition assays are also well-known in the art. See, e.g., U.S. Pat. No. 6,162,828, incorporated herein by reference in its entirety. In certain embodiments, the PFMK may be a cathepsin B inhibitor. The virus may be orthoreovirus or rotavirus, for example. The virus may be any type of pathogenic poxvirus, in certain embodiments. In certain particular embodiments, the virus is a reovirus. The reovirus may be a wild-type reovirus or an attenuated reovirus. In certain particular embodiments, the reovirus is an attenuated reoviruses. The attenuated reovirus may lack a wild-type reovirus S1 gene, n certain embodiments. The reovirus may be a variola virus or a monkeypox virus, for example. In certain embodiments, the Poxyiridae virus is a poxvirus. In certain embodiments, the Poxyiridae virus is a chordopoxyirinae virus. Chodopoxyirinae viruses include, for example, orthopoxvirus and leporipoxvirus. Non-limiting examples of orthopoxvirus include vaccinia virus, monkeypox virus, cowpox and variola virus. In certain embodiments, the Poxyiridae virus is a leporipoxvirus, such as myxoma virus.

The PFMK may be of any type described herein. In certain embodiments, the PFMK is Z-FA-FMK. In certain embodiments, a PFMK may inhibit viral replication by at least about 75%. In certain embodiments, a PFMK may inhibit viral replication by at least about 50%. In certain embodiments, a PFMK may inhibit viral replication by at least about 99%. In certain embodiments, a PFMK may inhibit viral replication by about 100%.

In certain embodiments, viral replication may be inhibited in a cell that is comprised in neural tissue. The cell may be comprised in cardiac muscle tissue. In certain embodiments, the cell is a cancer cell. The cancer cell may be of any type of cancer known in the art. In certain embodiments, the cancer cell is a lung cancer cell, a colon cancer cell, a pancreatic cell, a thyroid cell, a white blood cell, or a melanocyte. The cancer cell may be comprised in a carcinoma. Non-limiting examples of such carcinomas include melanoma, adenocarcinoma, squamous cell carcinoma, small cell carcinoma and oat cell carcinoma. The cancer cell may, in certain embodiments, be comprised in a sarcoma. Non-limiting examples of such sarcomas include fibrosarcoma, chondrosarcoma, osteosarcoma, hepatoma and neuroblastoma. The cancer cell may be comprised in a hematopoietic malignancy. Non-limiting examples of hematopoietic malignancies include lymphoma, leukemia, myeloma, myelodysplastic syndrome and myeloproliferative disorders.

In certain embodiments, the cell is persistently infected with a virus, as defined herein. A cell may be persistently infected with a reovirus.

Any cell discussed herein may be in vitro or in vivo, in certain embodiments. Moreover, any of the methods discussed herein may take place in vitro or in vivo, in certain embodiments. In certain in vitro methods, the cell may be comprised in a mixed cell population. A mixed cell population of the present invention may, in certain embodiments, comprise stem cells. The stem cells may be hematopoietic stem cells.

In certain in vivo methods, the cell may be comprised in a subject. A subject may be a mammal, for example, a human. A human may be a newborn, a child, or an adult. In certain embodiments, the human may be less than one year old. In certain embodiments, the human may be a pregnant human. In certain embodiments, the human is infected with a virus. The human may be infected with a reovirus. The infection stemming from the reovirus may be naturally occurring, such as an infection that causes diarrhea. The subject may have cancer. The subject having cancer may be undergoing anti-cancer therapy, or may be about to receive anti-cancer therapy, or may have recently completed anti-cancer therapy. The anti-cancer therapy may be of any type known in the art. In particular embodiments, the anti-cancer therapy is reoviral anti-cancer therapy. Such therapy is well-known in the art. In certain embodiments, the subject is immunocompromised, as defined herein.

A PFMK of the present invention may be comprised in a pharmaceutically acceptable composition. Such compositions are described in more detail herein.

In certain aspects, the present invention contemplates a method of inhibiting viral replication in a subject, comprising administering an effective amount of a PFMK to the subject. The virus may be, for example, a Reoviridae virus or a Poxyiridae virus. The administration of the PFMK may be via any method known in the art. Non-limiting examples of such methods of administration include intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, subcutaneously, subconjunctival, intravesicularlly, mucosally, intrapericardially, intraumbilically, intraocularally, orally, locally, via inhalation, via injection, via infusion, via continuous infusion, via localized perfusion bathing target cells directly, via a catheter, via a lavage, in cremes, in lipid compositions, or any combination thereof.

In certain embodiments of any method described herein, a virus of the present invention is further defined as a virus that exhibits less dependence on an exogenous cysteine protease inhibitor than reovirus or myxoma virus, or both. Methods of measuring cysteine protease activity in the context of viral infection are well-known in the art. See, e.g., Takahasi et al., 1999; Gotoh et al., 1984; Saville et al., 2002, each of which are incorporated herein by reference.

As used herein, “reovirus” refers to any virus classified in the Reoviridae family. Reoviridae is the largest and most diverse family in terms of the host range of its members which include, for example, Orthoreovirus, Orbivirus, Rotavirus, Coltivirus, Aquareovirus, Cypovirus, Fijivirus, Phytoreovirus, Oryzavirus, Idnoreovirus and Mycoreovirus. The genomes of these viruses comprise 10, 11, or 12 segments of dsRNA, each encoding one to three proteins (usually one) on only one of the complementary strands (Fields, 1996).

The name reovirus (respiratory and enteric orphan virus) is a descriptive acronym suggesting that these viruses, although not associated with any known disease state in humans, can be isolated from both the respiratory and enteric tracts (Sabin, 1959). The term “reovirus” refers to all viruses classified in the Reoviridae family, and is described in more detail below.

“Attenuated” reoviruses described herein include reoviruses that exhibit altered (i.e., increased or decreased in a statistically significant manner) infective, replicative and/or lytic properties toward or in a host cell, relative to levels of one or more such properties that are exhibited by known, naturally occurring or wild-type reoviruses. In certain embodiments, the attenuated reovirus will exhibit decreased infectivity, replicative ability and/or lytic potential, relative to a wildtype reovirus. Examples of such altered properties by which one may discern an attenuated reovirus as presently disclosed include various manifestations of viral cytopathic effects, for instance, the multiplicity of infection (MOI, the average number of virions that infect each cell) required for productive infection of a given host cell, the degree of host cell cytolysis induced by viral infection (further including apoptosis and/or necrosis), the titer of viruses released from a productively infected host cell following cytolytic viral replication, and other parameters by which those familiar with the art can determine viral activities toward host cells. Other indicia of cytopathic effects include altered host cell morphology, altered cell adhesion (to substrates such as extracellular matrix proteins or semisolid growth media, or to other cells), altered expression levels of one or more cellular genes, altered ability of host cells to replicate, and/or other alterations in cellular metabolic activity.

As used herein, a “peptidyl fluoromethylketone” (PFMK) is a fluoromethylketone-containing compound that contains two or more amino acids and reduces or inhibits viral replication by about or at least about 50%, about 55%, about 60%, about 65%, about 70%, about 75%, about 80%, about 85%, about 90%, about 95%, about 99%, or more, or about 100%, or any value or range in between. The amino acid may be any natural amino acid, any unnatural amino acid, or any combination thereof, as known by those of skill in the art. In certain aspects, a PFMK may be chemically modified and still retain the desired effects of the PFMK compound prior to the chemical modification. Such modified PFMKs may involve the addition, removal, or substitution of one or more chemical moieties on the parent PFMK molecule. Non-limiting examples of the types modifications that can be made to the compounds and structures disclosed herein include the addition or removal of peptide moiety, or lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, and phosphoryl groups, and halide substituents. Additional modifications can include an addition or a deletion of one or more atoms of the atomic framework, for example, substitution of an ethyl by a propyl, or substitution of a phenyl by a larger or smaller aromatic group. Alternatively, in a cyclic structure, heteroatoms such as N, S, or O can be substituted into the structure instead of a carbon atom.

As noted, the term “amino acid” refers to any of the naturally occurring amino acids, as well as synthetic analogs (e.g., D-stereoisomers of the naturally occurring amino acids, such as D-threonine) and derivatives thereof. α-Amino acids comprise a carbon atom to which is bonded an amino group, a carboxyl group, a hydrogen atom, and a distinctive group referred to as a “side chain.” Amino acids comprising an additional methylene group in their backbone are often called β-amino acids. The side chains of naturally occurring amino acids are well known in the art and include, for example, hydrogen (e.g., as in glycine), alkyl (e.g., as in alanine, valine, leucine, isoleucine, proline), substituted alkyl (e.g., as in threonine, serine, methionine, cysteine, aspartic acid, asparagine, glutamic acid, glutamine, arginine, and lysine), arylalkyl (e.g., as in phenylalanine and tryptophan), substituted arylalkyl (e.g., as in tyrosine), and heteroarylalkyl (e.g., as in histidine). Unnatural amino acids are also known in the art, as set forth in, for example, Williams (1989); Evans et al. (1990); Pu et al. (1991); Williams et al (1991); and all references cited therein. The present invention includes the side chains of unnatural amino acids as well.

A variety of amino protecting groups may be appended to a PFMK, such as to the N-terminus of a PFMK. Such protecting groups are well-known in the art, and non-limiting examples include a benzyloxycarbonyl (Z or Cbz) group, a 1-butoxycarbonyl (Boc) group and an N-morpholineurea group. Other such groups are listed in Greene and Wuts, 1999, incorporated herein in its entirety. Such protecting groups may be chemically modified as described above.

Yet other modifications to a PFMK may include the addition of substituents that target the resultant PFMK to a particular cellular compartment, such as an endosome or a lysosome. Such substituents are well-known in the art. Non-limiting examples of cellular compartment-targeting substituents include cyclodextrin and galactosyl carbohydrate groups. Such substituents may localize enhancement in concentration of the PFMK at the site of its putative activity, allowing for lower concentrations of the PFMK to be administered. Such substituents may also provide enhanced solubility, persistance and/or stability in vivo and/or in vitro. The present invention specifically contemplates PFMKs that comprise cellular compartment-targeting substituents.

Non-limiting, exemplary peptidyl fluoromethylketones (PFMKs) include Z-FA-FMK (N-benzyloxycarbonyl-Phe-Ala-fluoromethylketone), Mu-F-hF-FMK (N-morpholineurea-phenylalanyl-homophenylalanyl-fluoromethylketone), Z-VAD-FMK (Z-Val-Ala-Asp-fluoromethylketone), Z-VAD(OMe)-fluoromethylketone, Biotin-IETD-FMK, Biotin-Phe-Ala-FMK, Phe-Ala-Met-Asp(OMe)-Glu(OMe)-Val-Asp(OMe)-FMK, Mu-Leu-hF-FMK, Mu-Val-hF-FMK, Z-ATAD(OMe)-FMK, Z-Leu-Leu-Leu-FMK, Z-Leu-Leu-Tyr-FMK, Z-Phe-Phe-FMK and others.

In certain aspects, a PFMK may be characterized as follows, wherein R represents a di-, tri-, tetra-, penta-, or hexa-peptide:

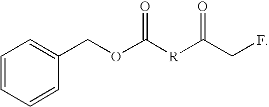

As used herein, “persistently infected with a virus” refers to a viral infection status in which population of cells are partially infected with a virus. See, e.g., Kim et al., Oncogene, 2007, incorporated herein by reference in its entirety.

As used herein, “immunocompromised” refers to a subject (e.g., animal, patient) with an immune system rendered deficient relative to a normal subject by, for example, breeding, an immunodeficiency disorder or other disease, administration of an immunosuppressive agent, administration of chemotherapy, or exposure to radiation. Examples of immunocompromised subjects include mammals suffering from viral infections such as AIDS, influenza, cancer patients receiving chemotherapy and/or radiotherapy, transplant patients receiving an antirejection agent, and/or patients that have received toxic chemicals, metals, and/or radiation exposure.

The terms “contacted” and “exposed,” when applied to a cell, tissue or organism, are used herein to describe the process by which a compound, such as a PFMK, e.g., Z-FA-FMK, is delivered to a target cell, tissue, or organism or is placed in direct juxtaposition with the target cell, tissue, or organism.

As used herein, “cellular composition” means a composition comprising cells. The composition may contain non-cellular matter. For example, whole blood is a cellular composition which contains plasma, platelets, hormones and other non-cellular matter in addition to cells such as erythrocytes and leukocytes. A cellular composition may contain cells of various types, origin, or organization. For example, tissues and organs which contain different cell types arranged in defined structures are considered cellular compositions. Cellular compositions which contain more than one cell type are referred to as “mixed cellular compositions.”

The term “effective,” as that term is used in the specification and/or claims (e.g., “an effective amount,” means adequate to accomplish a desired, expected, or intended result.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

Throughout this application, the term “about” is used to indicate that a value includes the inherent variation of error for the device or the method being employed to determine the value, or the variation that exists among the study subjects. For example, “about” can be within 10%, preferably within 5%, more preferably within 1%, and most preferably within 0.5%.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

It is contemplated that any embodiment discussed in this specification can be implemented with respect to any compound, method, or composition of the invention, and vice versa.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1E: PFMKs (Z-FA-FMK, Mu-F-hF-FMK, and Z-VAD-FMK) mediated inhibition of reovirus infection. (FIG. 1A) HT1080 and mouse embryonic stem cells (D3) were either mock infected or infected with either reovirus (MOI of 40) alone or reovirus with Z-FA-FMK (20 μM) in culture medium. After 48 hr of culture, viral cytopathic morphology was photographed. (FIG. 1B) Viral antigens were detected by western blotting and FACS analysis using reovirus antiserum. (FIG. 1C) HTR1 cells grown to 70% confluency were either mock treated or treated with Z-FA-FMK (20 μM) in culture medium, and passaged for 2 weeks. Then, cell lysates were prepared and analyzed by western blotting using reovirus antiserum. (FIG. 1D) HT1080 cells grown to 50% confluency were either mock infected or infected with either reovirus (MOI of 40) alone or reovirus with Z-FA-FMK at indicated doses (0 μM, 0.2 μM, 2 mM, and 20 μM) in culture medium. After 48 hr of viral infection, cells were then examined by FACS analysis using rabbit polyclonal reovirus-antiserum and FITC-conjugated secondary goat anti-rabbit IgG for reoviral antigen detection as described in the Examples. Mock: mock infection. (FIG. 1E) Other peptidyl fluoromethylketones (PFMKs) that inhibited reovirus-induced cytopathogenicity. Mu-F-hF-FMK (N-Morpholineurea-phenylalanyl-homophenylalanylfluoromethylketone) and Z-VAD-FMK (Z-Val-Ala-Asp(OCH₃)-fluoromethylketone) (20 μM each) were treated in either the presence or absence of reovirus (MOI of 20) on HT1080 monolayers. After 48 hr of culture, viral cytopathic morphology was photographed.

FIGS. 2A-2B: Electron microscopic analysis of reovirus infected HT1080 cells in the presence or absence of Z-FA-FMK treatment. HT1080 cells grown to 50-70% confluency were infected with reovirus at MOI of 40, then either Z-FA-FMK (20 μM) or control medium was added in the culture medium. After 48 hr culture, cells were prepared for electron microscopic examination as described in the Examples. (FIG. 2A) HT1080 infected with reovirus in the absence of Z-FA-FMK showing scattered virions and viral inclusions (factory) (arrows) in the perinuclear region. Scale represents 500 nm×10,000 (above). Crystalline array of viral inclusions shows full or empty viral particles (bottom). Scale represents 100 nm×80,000 (FIG. 2B) HT1080 infected with reovirus in the presence of Z-FA-FMK showing scattered reovirus virion particles (arrows) but no reoviral inclusions in the perinuclear region. Scale represents 500 nm×10,000 (above). Scattered virions with empty shell (arrows) are present in perinuclear region (bottom). Scale represents 200 nm×40,000.

FIGS. 3A-3B: Z-FA-FMK does not affect influenza A virus or HIV-1 virus. (FIG. 3A) Mv1Lu cells were either mock-infected or infected with Flu type A (A/New Cale/H1N1) strain with Z-FA-FMK (0 μM, 20 μM) in culture medium. After 4 days, cells were fixed/permeablized for FACS analysis using FITC-conjugated Flu type A antiserum. (FIG. 3B) Ghost-CXCR4 cells were either mock-infected or infected with CXCR4 using HIV type 1 strain (NL4-3) with Z-FA-FMK (0 μM, 20 μM) in culture medium. After 3 days, cells were photographed and examined under a fluorescence microscope. The Ghost-CXCR4 cell line carries the HIV long terminal repeat-driven green fluorescence protein (GFP) gene, which becomes activated upon infection with HIV and thus became fluorescent (Vodros and Fenyo, 2005).

FIGS. 4A-4B: Z-FA-FMK blocks reovirus infection in vivo. (FIG. 4A) HT1080 cells were injected subcutaneously into hind flanks of SCID mice. After palpable tumors were established (11 days after implantation), tumors were injected with Live reovirus (L Reo; n=5 tumors), UV-inactivated reovirus (D Reo; n=5 tumors), Z-FA-FMK (FA; n=5 tumors) and Live reovius suspended in Z-FA-FMK solution (Reo+FA; n=5 tumors). Tumor growth was measured externally using calipers. Z-FA-FMK was dissolved in DMSO and administered as 0.02 mg suspended in PBS intratumorally per mouse everyday up to 7 days post viral injection, and every 2 days until completion of the experiment. (FIG. 4B) Tumors and heart tissues were taken from the mice at 27 days post implantation (16 days post-viral infection). Paraffin sections of tumors and heart tissues were analyzed by indirect immunohistochemical staining using reovirus antiserum as described in the Examples. Deep purple staining of tumors represents reoviral protein positive and the sections were counterstained with hematoxylin. Brown staining of heart tissues represents reoviral protein positive and the sections were counterstained with methyl green.

FIGS. 5A-C: Z-FA-FMK suppresses myxoma virus foci development. (FIG. 5A) BGMK cells were infected with Myx-GFP at MOI of 0.5 in the presence of Z-FA-FMK. At 2 days post-infection, GFP expression level of BGMK monolayers were photographed. Myxoma virus foci development is significantly suppressed as Z-FA-FMK dosages are increased. (FIG. 5B) BGMK cells were infected with Myx-GFP at MOI of 0.01 in the presence of Z-FA-FMK. At 3 days post-infection, GFP foci development was photographed. Z-FA-FMK effectively suppressed GFP foci development. (FIG. 5C) BGMK cells were infected with Myx-GFP at MOI of 3 in the presence of Z-FA-FMK. At 1 day post-infection, GFP expressing cells were photographed. At high MOI challenge, Z-FA-FMK does not inhibit myxoma infectivity as shown by GFP expression level, indicating that Z-FA-FMK only suppresses myxoma virus foci development. Myx-GFP: GFP expressing myxoma virus (strain Lausanne), which contains a green fluorescent protein (GFP) cassette driven by a synthetic Vaccinia virus early/late promoter. MOI: Multiplicity of infection. BGMK cells: Buffalo green monkey kidney cells.

The patent or application file contains at least one drawing executed in color. Copies of this patent or patent application publication with color drawing(s) will be provided by the Office upon request and payment of the necessary fee.

### DESCRIPTION OF ILLUSTRATIVE EMBODIMENTS

The present invention relates to the discovery that certain PFMKs inhibit viral replication. In certain aspects, the virion cysteine protease activity or virion maturation activity of a variety of viruses may be affected by the administration of a PFMK. Such viruses include members of the Reoviridae and Poxyiridae families. These and other aspects of the invention are described in greater detail below.

### 1. CATHEPSINS AND CATHEPSIN INHIBITORS

Cathepsins belong to the papain superfamily of lysosomal cysteine proteases, which comprise roughly a dozen members that are distinguished by their structure and which proteins they cleave. These proteins play a key role in mammalian cellular turnover, such as bone resorption. For example, cathepsin K, which is characterized by its high specificity for kinins, is involved in bone resorption. Accordingly, cathepsins have been associated with osteoporosis as well as cancer (Nomura, 2005), stroke (Lipton, 1999), arthritis and Alzheimer's disease (Pham, 2005) as well as muscular dystrophy, bronchitis and emphysema. See, e.g., U.S. Pat. No. 6,605,589 and references therein. For example, Cathepsin L is implicated in tumor invasiveness and the formation of metastases as well as in the development of inflammatory diseases (e.g., arthritis). Cathepsin C also plays an immunological role. Cathepsin B is implicated in the breakdown of proteins which cause amyloid plaque—the root of Alzheimer's symptoms—and may be a pivotal player in the body's natural defense against this disease. Other cathepsins include cathepsins A, D, E, F, G, H, O, S, X, V and Z.

In view of their roles in many physiological processes, increased interest in cathepsin inhibitors has been generated as potential therapeutic targets, such as cathepsin K or cathepsin L for osteoporosis and cathepsin S for immune modulation. See Vasiljeva et al., 2007; Dilakian and Tsvetkova, 2005. Cathepsin inhibitors are generally known in the art. For example, the cathepsin K inhibitor balicatib passed Phase II clinical trials in 2005.

Peptidyl fluoromethylketones (PFMKs) are cell-penetrable small peptide molecules with a low toxicity profile, and have been shown to be irreversible inhibitors of some members of the cathepsin enzyme family and cysteine proteases, and may offer a potential therapy for the treatment of osteoarthritis (Ahmed et al., 1992; Rasnick, 1985; Esser et al., 1993). Certain PFMKs with amino acids phenylalanine and alanine in the P2 and P1 positions, respectively, have been shown to be irreversible inhibitors of some members of the cathepsin enzyme family (Ahmed et al., 1992; Rasnick, 1985). Other cathepsin inhibitors are described in the patent literature. See, e.g., U.S. Pat. Nos. 6,605,589; 6,596,715; 6,534,498; 6,124,257; and 5,698,519. Accordingly, in certain embodiments, a PFMK may be a cathepsin inhibitor.

PFMKs can be obtained via synthetic methods known in the art or from commercial sources (e.g., Sigma-Aldrich Co., Milwaukee, Wis.; Axxora Platform, San Diego, Calif.; BIOMOL International, Plymouth Meeting, Pa.). For example, amino-acid based inhibitors may be prepared as described in Stewart and Young, “Solid Phase Peptide Synthesis”, 2nd ed., Pierce Chemical Co., Rockford, Ill. (1984); Gross, Meienhofer, Udenfriend, Eds., “The Peptides: Analysis, Synthesis, Biology”, Vol 1, 2, 3, 5 and 9, Academic Press, New York, 1980-1987; Bodanszky, “Peptide Chemistry: A Practical Textbook”, Springer-Verlag, New York (1988); and Bodanszky, et al., “The Practice of Peptide Synthesis”, Springer-Verlag, New York (1984), the disclosures of which are hereby incorporated by reference. Processes for incorporating a fluoromethylketone group into a peptide are also well-known. See, e.g., U.S. Pat. Nos. 5,210,272 and 5,344,939, each of which are incorporated herein by reference. Assays to determine whether a particular compound inhibits one or more cathepsins are also well known. See, e.g., U.S. Pat. Nos. 6,605,589; 6,596,715; 6,534,498; 6,124,257; and 5,698,519, each of which are incorporated herein by reference.

As mentioned above, certain PFMKs with the amino acids phenylalanine and alanine in the P2 and P1 positions, respectively, have been shown to be irreversible inhibitors of some cathepsins (Ahmed et al., 1992; Rasnick, 1985). In particular, N-benzyloxycarbonyl-Phe-Ala-fluoromethylketone (Z-FA-FMK) was found to be a potent inactivator of cellular cathepsin B and binds tightly to the active site of the enzyme. Once bound to the active site, the cysteine residue in the active site is alkylated by the fluoromethylketone group, which irreversibly blocks its proteolytic activity. Assays to determine whether a compound is a cathepsin B inhibitor are well-known in the art. See, e.g., U.S. Pat. No. 5,068,354 and Barrett and Kirschke (1981) each of which are incorporated herein by reference. A working example is provided in Example 5 below. Examples 1, 2 and 4 below describe in vitro and in vivo methods for determining whether such a compound also affects viral replication.

It has been previously shown that cathepsin B plays a role in a proteolytic disassembly of viral outer-capsid proteins leading to proteolytic activation of reovirus virion and pharmacological inhibition (CA-074Me) of cellular cathepsin B contributes to decreased viral yields yet not a complete blockage of viral yield (Ebert et al., 2002). Therefore, it is likely that Z-FA-FMK-mediated complete blockage of the viral replication occurs via a different mechanism in addition to inhibition of the proteolytic disassembly of viral outer-capsid proteins.

In certain embodiments, PFMKs may, instead of irreversibly inactivating a cathepsin or other enzyme involved in viral activity, only transiently inactivate such an enzyme. Such transient inactivation may be reversible upon, for example, flushing out of the compound, or upon decay of the compound. Such reversibility may be desirable if a PFMK is used at higher concentrations and/or for extended periods of time.

The inventors have surprisingly discovered that certain PFMKs, such as Z-FA-FMK, likely affect unknown viral/host factors involved in virion packaging and factory development during certain virus infection. For example, during reovirus infection, it has been suggested that muNS, sigmaNS and sigma 3 play a role in the reovirus RNA packaging process (Nibert et al., 1996). Thus, it is possible that certain PFMKs specifically affect the normal function of these packaging related reoviral proteins. Accordingly, other PFMKs that affect pathways other than the proteolytic disassembly of viral outer-capside proteins to reduce or block viral replication are specifically contemplated by the present invention. Other viruses that may be affected in this manner by PFMKs include members of the Rotoviridae and Poxyiridae families, such as rotavirus and smallpox, respectively.

### 2. VIRUSES

Certain viruses are susceptible to inhibition by the PFMKs disclosed herein. In certain embodiments, the virus is an enveloped virus. In certain embodiments, the virus is a non-enveloped virus. Select viruses are described in more detail below.

A. Reoviridae

The Reoviridae are a family of viruses that include some viruses that affect the gastrointestinal system and some that caue respiratory infections. The genetic material of viruses in this family is double-stranded RNA. The name Reoviridae is derived from respiratory eneteric orphan viruses. The term “orphan virus” means a virus that is not associated with any known disease. Even though Reoviridae have been identified with various diseases, the original name is still used.

Reovirus infection occurs often in humans, but most cases are mild or subclinical. The virus can be readily detected in feces, and many also be recovered from pharyngeal or nasal secretions, urine, cerebrospinal fluid, and blood. Despite the ease of finding Reovirus in clinical specimens, their role in human disease or treatment is still uncertain.

Reoviruses are non-enveloped and have an iscohedral capsid (T-13). It is composed of an outer and inner shell—a double protein shell. The genome consists of three segments, which are, L, M and S corresponding to their size. S segments being the smallest and L being the largest. Segments range from about 3.9 kbp-1 kbp. The L segment encodes for λ proteins, the M segment encodes for μ proteins and the S segment encodes for σ proteins. The different segments are transcribed and translated at different rates.

Non-limiting examples of Rotoviridae genus members include Orthoreovirus, Orbivirus, Rotavirus, Coltivirus, Aquareovirus, Cypovirus, Fijivirus, Phytoreovirus, Oryzavirus, Idnoreovirus and Mycoreovirus. Specific species include, but are not limited to, mammalian orthoreovirus, bluetongue virus, rotavirus A, colorado tick fever virus, aquareovirus A, cypovirus 1, Fiji disease virus, rice dwarf virus, rice ragged stunt virus, idnoreovirus I and mycoreovirus 1.

1. Reovirus

As discussed above, reovirus is generally benign in human infections, but reovirus-induced pathogenesis may occur in immunocompromised hosts (Kim et al., 2007; Loken et al., 2004). Reovirus also adversely affects development of rat and murine embryos, retarding development and inhibiting blastocytst formation. (Heggie and Gaddis, 1979; Priscott, 1983). The present invention concerns the use of PFMKs to modulate viral replication activity, such as during reovirus anti-cancer therapy. Reovirus anti-cancer regimens are known in the art. See, e.g., U.S. Pat. No. 7,163,678; Norman and Lee (2005), incorporated herein by reference in their entirety.

As used herein, “reovirus” refers to any virus classified in the reovirus genus. The name reovirus (respiratory and enteric orphan virus) is a descriptive acronym suggesting that these viruses, although not associated with any known disease state in humans, can be isolated from both the respiratory and enteric tracts (Sabin, 1959). The term “reovirus” refers to all viruses classified in the reovirus genus.

The human reovirus consists of three serotypes: type 1 (strain Lang or T1L), type 2 (strain Jones, T2J) and type 3 (strain Dearing or strain Abney, T3D). The three serotypes are easily identifiable on the basis of neutralization and hemagglutinin-inhibition assays (see, for example, Fields et al., 1996).

The reovirus may be naturally occurring or modified. The reovirus is “naturally-occurring” when it can be isolated from a source in nature and has not been intentionally modified by humans in the laboratory. For example, the reovirus can be from a “field source,” that is, from a human who has been infected with the reovirus.

The reovirus may be modified but still capable of lytically infecting a mammalian cell having an active ras pathway. The reovirus may be chemically or biochemically pretreated (e.g., by treatment with a protease, such as chymotrypsin or trypsin) prior to administration to the proliferating cells. Pretreatment with a protease removes the outer coat or capsid of the virus and may increase the infectivity of the virus. The reovirus may be coated in a liposome or micelle (Chandran and Nibert, 1998) to reduce or prevent an immune response from a mammal which has developed immunity to the reovirus. For example, the virion may be treated with chymotrypsin in the presence of micelle forming concentrations of alkyl sulfate detergents to generate a new infectious subvirion particle.

The reovirus may be a recombinant reovirus from two or more types of reoviruses with differing pathogenic phenotypes such that it contains different antigenic determinants, thereby reducing or preventing an immune response by a mammal previously exposed to a reovirus subtype. Such recombinant virions can be generated by co-infection of mammalian cells with different subtypes of reovirus with the resulting resorting and incorporation of different subtype coat proteins into the resulting virion capsids.

The reovirus may be an attenunated reovirus, such as that described in International Patent Application No. PCT/US2006/029881, filed Jul. 31, 2006, incorporated herein by reference in its entirety. Such an attenuated reovirus may comprise a reovirus genome that lacks a wild-type reovirus S1 gene, wherein the attenuated reovirus is derived from a host cell culture that has been persistently infected with a reovirus. In certain embodiments, the attenuated reovirus is derived from a human reovirus, and in a further embodiment the human reovirus is selected from human reovirus Type 1, human reovirus Type 2 and human reovirus Type 3. In a still further embodiment, the human reovirus is selected from human reovirus Type 1 strain Lang, human reovirus Type 2 strain Jones, human reovirus Type 3 strain Dearing and human reovirus Type 3 strain Abney. In one further embodiment of the above described attenuated reovirus, the host cell is a mammalian host cell. In another embodiment, the mammalian host cell is a human host cell.

In another embodiment, there is provided an attenuated reovirus, comprising a reovirus genome that lacks a wild-type reovirus S1 gene, wherein said wild-type reovirus S1 gene comprises a polynucleotide sequence that is at least 90% identical to a sequence selected from SEQ ID NO:1 (T1L, M35963), SEQ ID NO:3 (T2J, M35964), SEQ ID NO:5 (T3D, X01161) and SEQ ID NO:7 (T3A, L37677). In another embodiment, there is provided an attenuated reovirus, comprising a reovirus genome that lacks a reovirus S1 gene which is capable of encoding a reovirus (1 capsid protein having an amino acid sequence selected from (i) an amino acid sequence that is greater than 10% identical to the sequence set forth in SEQ ID NO:2, 4, 6 or 8, (ii) an amino acid sequence that is greater than 20% identical to the sequence set forth in SEQ ID NO: 2, 4, 6 or 8, (iii) an amino acid sequence that is greater than 40% identical to the sequence set forth in SEQ ID NO: 2, 4, 6 or 8, (iv) an amino acid sequence that is greater than 50% identical to the sequence set forth in SEQ ID NO: 2, 4, 6 or 8, (v) an amino acid sequence that is greater than 70% identical to the sequence set forth in SEQ ID NO: 2, 4, 6 or 8, (vi) an amino acid sequence that is greater than 90% identical to the sequence set forth in SEQ ID NO: 2, 4, 6 or 8, and (vii) an amino acid sequence that is greater than 95% identical to the sequence set forth in SEQ ID NO: 2, 4, 6 or 8.

In another embodiment, there is provided an attenuated reovirus comprising a mutated reovirus S1 gene that is incapable of encoding a reovirus σ1 capsid protein having an amino acid sequence selected from (i) an amino acid sequence that is greater than 10% identical to the sequence set forth in SEQ ID NO: 2, 4, 6 or 8, (ii) an amino acid sequence that is greater than 20% identical to the sequence set forth in SEQ ID NO: 2, 4, 6 or 8, (iii) an amino acid sequence that is greater than 40% identical to the sequence set forth in SEQ ID NO: 2, 4, 6 or 8, (iv) an amino acid sequence that is greater than 50% identical to the sequence set forth in SEQ ID NO: 2, 4, 6 or 8, (v) an amino acid sequence that is greater than 70% identical to the sequence set forth in SEQ ID NO: 2, 4, 6 or 8, (vi) an amino acid sequence that is greater than 90% identical to the sequence set forth in SEQ ID NO: 2, 4, 6 or 8, and (vii) an amino acid sequence that is greater than 95% identical to the sequence set forth in SEQ ID NO: 2, 4, 6 or 8.

In another embodiment, there is provided an attenuated reovirus, comprising a replication-competent reovirus virion that comprises a heritable mutant reovirus S1 gene, wherein said mutant reovirus S1 gene comprises one or a plurality of mutations in a polynucleotide sequence as set forth in SEQ ID NO: 1, 3, 5 or 7, and wherein the mutant reovirus gene is incapable of encoding at least one reovirus σ1 capsid protein that comprises an amino acid sequence selected from (i) an amino acid sequence that is greater than 10% identical to the sequence set forth in SEQ ID NO: 2, 4, 6 or 8, (ii) an amino acid sequence that is greater than 20% identical to the sequence set forth in SEQ ID NO: 2, 4, 6 or 8, (iii) an amino acid sequence that is greater than 40% identical to the sequence set forth in SEQ ID NO: 2, 4, 6 or 8, (iv) an amino acid sequence that is greater than 50% identical to the sequence set forth in SEQ ID NO: 2, 4, 6 or 8, (v) an amino acid sequence that is greater than 70% identical to the sequence set forth in SEQ ID NO: 2, 4, 6 or 8, (vi) an amino acid sequence that is greater than 90% identical to the sequence set forth in SEQ ID NO: 2, 4, 6 or 8, and (vii) an amino acid sequence that is greater than 95% identical to the sequence set forth in SEQ ID NO: 2, 4, 6 or 8. In certain further embodiments, the one or a plurality of mutations comprises at least one mutation selected from a nucleotide substitution, a nucleotide deletion and a nucleotide insertion.

In another embodiment, there is provided an attenuated reovirus, comprising a replication-competent reovirus virion that lacks a detectable reovirus σ1 capsid protein.

In another embodiment, there is provided an attenuated reovirus, comprising a replication-competent reovirus virion that lacks a detectable reovirus σ1 capsid protein having a polypeptide sequence as set forth in SEQ ID NO: 2, 4, 6 or 8. In another embodiment, there is provided an attenuated reovirus, comprising a replication-competent reovirus virion that lacks a detectable reovirus σ1 capsid protein having a polypeptide sequence that is selected from (i) a polypeptide sequence that is at least 50% identical to the sequence set forth in SEQ ID NO: 2, 4, 6 or 8, and (ii) a polypeptide sequence that is at least 20% identical to the sequence set forth in SEQ ID NO: 2, 4, 6 or 8. In certain further embodiments, the above described attenuated reovirus lacks a wild-type reovirus S4 gene.

Certain embodiments of the invention provide an attenuated reovirus, comprising a replication-competent reovirus virion that lacks a detectable reovirus σ1 capsid protein having a polypeptide sequence that is at least 10% identical to the sequence set forth in SEQ ID NO: 2, 4, 6 or 8. Certain further embodiments of the above described attenuated reovirus provide such an attenuated reovirus that exhibits a decreased level of at least one detectable cytopathic effect toward a non-malignant cell relative to the level of the detectable cytopathic effect that is exhibited toward the non-malignant cell by a non-attenuated reovirus. In certain further embodiments, the non-malignant cell is selected from (i) a differentiated normal cell that comprises at least one of a cardiac myocyte, a pancreatic cell and an endothelial cell, and (ii) an undifferentiated stem cell that comprises at least one of an embryonic stem cell and a neural stem/progenitor cell. In certain other further embodiments, the detectable cytopathic effect comprises at least one detectable cytopathic effect that is selected from apoptosis, necrosis, cytolytic viral replication, altered cell morphology, altered cell adhesion, altered cellular gene expression, altered cellular replication, and altered cellular metabolic activity.

2. Rotavirus

As discussed above, rotaviruses, which are members of the Reoviridae family, are the leading cause of severe, life-threatening viral gastroenteritis in infants and animals (Kapikian et al., 1996) and are associated with sporadic outbreaks of diarrhea in elderly (Halvorsrud, 1980) and immunocompromised patients (Holzel et al., 1980). Indeed, rotaviruses are the most common cause of severe diarrhea in children.

Rotavirus has a characteristic wheel-like appearance when viewed by electron microscopy (the name rotavirus is derived from the Latin rota, meaning “wheel”). Rotaviruses are nonenveloped, double-shelled viruses. The genome is composed of 11 segments of double-stranded RNA, which code for six structural and five nonstructural proteins.

The present invention specifically contemplates the use of PFMKs to treat naturally occurring viral infections. As such, PFMKs of the present invention may be used to treat naturally occurring rotaviral infections, such as diarrhea-particularly, but not exclusively, diarrhea that occurs in small children.

B. Poxyiridae

Poxviruses are the largest known animal viruses, with ˜200 distinct genes (Moss, 2001). They are DNA viruses that replicate entirely in the cytoplasm. Thus, a subset of their gene products carries out the functions that are essential for the viruses to be independent of the host-cell nucleus. The other viral gene products use or modulate an astonishingly wide array of host-cell and immune-system processes. Poxviruses infect most vertebrates and invertebrates, causing a variety of diseases of veterinary and medical importance. Myxoma virus is a leporipoxvirus that infects rabbits and causes myxomatosis. The one large family (Poxyiridae) has two main subfamilies, the chordopoxyirinae, which infect vertebrates, and the entomopoxyirinae, which infect insects. Chordopoxyirinae comprises orthopoxvirus, leporipoxvirus and others. Orthopoxvirus comprises vaccinia virus, cowpox virus, monkeypox virus, variola virus and others. Leporipoxvirus comprises myxoma virus, hare fibroma virus, rabbit fibroma virus and squirrel fibroma virus.

The poxvirus replication cycle is a complex sequence of cytoplasmic events that begins with binding to the cell surface and subsequent fusion of virus and mammalian cell membranes. The intracellular replication cycle has been most well studied for vaccinia virus, which is the vaccine strain that was used to eradicate smallpox, but the essential features are highly conserved amongst other poxviruses (Moss, 2001). Two distinct infectious virus particle types—the intracellular mature virus (IMV) and the extracellular enveloped virus (EEV)—can initiate the infectious cycle (Smith et al., 2002). So far, several virion proteins have been shown to be crucial for binding of the virion to the cell surface, but the cell determinants of binding are thought to be ubiquitously expressed glycosaminoglycans or components of the extracellular matrix (McFadden, 2005). After binding, the fusion event between the virion and the host cell membranes is still poorly understood, but at least one conserved virion protein (VV-A28) has been linked to this fusion/entry event that ultimately releases the virion core structure into the cytoplasm (Senkevich et al., 2004).

The I7 cysteine protease, encapsidated in the virus particle, processes the major virion core proteins at a cleavage motif, AlaGly↑X. A loss in functionality of the enzyme leads to arrest of virion morphogenesis at a point after the formation of spherical immature particles. This protein of 432 aa is 99% identical in variola and monkeypox viruses; it appears to have only a very distant relationship to the cellular SUMO-specific protease Ulp1 and to adenovirus and African swine fever virus (ASFV) proteases (Harrison et al., 2004).

### 3. PHARMACEUTICAL FORMULATIONS AND ADMINISTRATION THEREOF

A. Pharmaceutical Formulations and Routes for Administration to Subjects

Pharmaceutical compositions of the present invention comprise an effective amount of one or more candidate substance or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one candidate substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The candidate substance may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, subcutaneously, subconjunctival, intravesicularlly, mucosally, intrapericardially, intraumbilically, intraocularally, orally, locally, via inhalation (e.g., aerosol inhalation), via injection, via infusion, via continuous infusion, via localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of a PFMK. In other embodiments, the PFMK may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof.

The candidate substance may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine, or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. It may be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in certain embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the candidate substance is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. In certain embodiments, carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar, or both.

Additional formulations which are suitable for other modes of administration include suppositories. Suppositories are solid dosage forms of various weights and shapes, usually medicated, for insertion into the rectum, vagina, or urethra. After insertion, suppositories soften, melt or dissolve in the cavity fluids. In general, for suppositories, traditional carriers may include, for example, polyalkylene glycols, triglycerides, or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, certain methods of preparation may include vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin, or combinations thereof.

B. Combination Therapy

In order to increase the effectiveness of a PFMK of the present invention, the compounds of the present invention may be combined with traditional drugs. It is contemplated that this type of combination therapy may be used in vitro or in vivo. In a non-limiting example, an anti-cancer agent may be used in combination with a PFMK. The anti-cancer agent may be a reoviral anti-cancer agent, and the reoviral anti-cancer agent may be used in combination with another anti-cancer agent, each of which may be used in combination with a PFMK of the present invention. In another non-limiting example, an anti-viral agent may be used in combination with a PFMK.

More generally, agents of the present invention would be provided in a combined amount with an effective amount of an anti-cancer agent to reduce or block viral replication. This process may involve contacting the cell(s) with the agents at the same time or within a period of time wherein separate administration of the substances produces a desired therapeutic benefit. This may be achieved by contacting the cell, tissue or organism with a single composition or pharmacological formulation that includes two or more agents, or by contacting the cell with two or more distinct compositions or formulations, wherein one composition includes one agent and the other includes another.

The compounds of the present invention may precede, be co-current with and/or follow the other agents by intervals ranging from minutes to weeks. In embodiments where the agents are applied separately to a cell, tissue or organism, one would generally ensure that a significant period of time did not expire between the time of each delivery, such that the agents would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the candidate substance. In other aspects, one or more agents may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 1, about 2, about 3, about 4, about 5, about 6, about 7 or about 8 weeks or more, and any range derivable therein, prior to and/or after administering the candidate substance.

Various combination regimens of the agents may be employed. Non-limiting examples of such combinations are shown below, wherein a PFMK is “A” and a second agent, such as an anti-cancer agent, is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

1. Anti-Cancer Agents

An “anti-cancer” agent is capable of negatively affecting cancer in a subject, for example, by killing one or more cancer cells, inducing apoptosis in one or more cancer cells, reducing the growth rate of one or more cancer cells, reducing the incidence or number of metastases, reducing tumor size, inhibiting tumor growth, reducing the blood supply to a tumor or one or more cancer cells, promoting an immune response against one or more cancer cells or a tumor, preventing or inhibiting the progression of a cancer, or increasing the lifespan of a subject with a cancer. Anti-cancer agents are well-known in the art and include, for example, chemotherapy agents (chemotherapy), radiotherapy agents (radiotherapy), a surgical procedure, immune therapy agents (immunotherapy), genetic therapy agents (gene therapy), reoviral therapy, hormonal therapy, other biological agents (biotherapy), and/or alternative therapies.

2. Anti-Viral Agents

An “anti-viral” agent is capable of inhibiting viral infection of cells. Many examples of antiviral compounds that can be used in combination with compounds of the present invention and are known in the art including, but are not limited to: rifampicin, nucleoside reverse transcriptase inhibitors (e.g., AZT, ddI, ddC, 3TC, d4T), non-nucleoside reverse transcriptase inhibitors (e.g., Efavirenz, Nevirapine), protease inhibitors (e.g., lopinavir, amprenavir, indinavir, ritonavir, and saquinavir), idoxuridine, cidofovir, acyclovir, ganciclovir, zanamivir, amantadine, and Palivizumab. Other examples of anti-viral agents include but are not limited to acemannan; acyclovir; acyclovir sodium; adefovir; alovudine; alvircept sudotox; amantadine hydrochloride; aranotin; arildone; atevirdine mesylate; pyridine; cidofovir; cipamfylline; cytarabine hydrochloride; delavirdine mesylate; desciclovir; didanosine; disoxaril; rdoxudine; enviradene; enviroxime; famciclovir; famotine hydrochloride; fiacitabine; fialuridine; fosarilate; foscamet sodium; fosfonet sodium; ganciclovir; ganciclovir sodium; idoxuridine; kethoxal; lamivudine; lobucavir; memotine hydrochloride; methisazone; nevirapine; penciclovir; pirodavir; ribavirin; rimantadine hydrochloride; saquinavir mesylate; somantadine hydrochloride; sorivudine; statolon; stavudine; tilorone hydrochloride; trifluridine; valacyclovir hydrochloride; vidarabine; vidarabine phosphate; vidarabine sodium phosphate; viroxime; zalcitabine; zidovudine; and zinviroxime.

### 4. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The following materials and methods were used for the experiments described in Examples 1-4:

### Cell Lines, Viruses and Z-FA-FMK

Human fibrosarcoma cell line (HT1080) and ES-D3 mouse embryonic stem cell line were purchased from the American Type Culture Collection (CRL-11632) and the latter were maintained in their undifferentiated state with 1×10⁶ U/ml of Leukemia Inhibitory Factor in media made according to ATCC protocols. ES-D3 cells were plated in 6 well plates at 4.8×10⁵ cells per well and grown at 37° C. at 95% humidity and 5% CO₂. To establish persistently infected cells with reovirus, reovirus susceptible HT1080 cells were infected with WT reovirus at MOI of 20, then the few surviving cells were maintained until they reached confluency. The surviving cells were subsequently re-infected 2-3 times for 3-7 weeks to select virally resistant cells. Initially these surviving cells were reluctant to grow and sometimes underwent crisis resulting in few surviving cells (Ahmed and Fields, 1982) but eventually they became established cell lines. From the surviving HT1080 cells, several subclones were obtained by serial dilution. Clone HTR1 (HT1080 virally Resistant clone 1) was used for this study. The HTR1 cells were persistently infected and maintained for more than 48 months (Kim et al., Oncogene). The Dearing strain of reovirus serotype 3 used in these studies was propagated in suspension cultures of L929 cells and purified according to previously established methods (Smith et al., 1969) with the exception that β-mercaptoethanol was omitted from the extraction buffer. For reovirus titration, HEK 293 cells were plated in 6-well plates at 2×10⁵ cells per well. After 2 hr of adsorption at 37° C., the inoculum was removed. Cell monolayers were then covered with 1% agar and fresh medium. Plaques were counted 5-7 days after infection. Mv1Lu cells were purchased from ATCC and maintained according to ATCC protocols. A/New Calcdonia/20/99-like virus (H1N1) was isolated from a respiratory specimen of a patient with acute influenza-like symptoms. The virus was isolated in primary rhesus monkey fibroblastic cells and subtyping was undertaken by HI serotyping and nucleic acid sequencing at the Provincial Laboratory for Public Health (Microbiology) in Alberta and the National Microbiology Laboratory (Winnipeg). Subtyping studies confirmed the virus is similar to that included in the current inactivated influenza virus vaccine. Ghost-CXCR4 cells obtained from the AIDS Reference and Reagent Program were maintained in a media containing high glucose DMEM (90%) and fetal bovine serum (10%) supplemented with G418 (500 μg/ml), hygromycin (100 μg/ml), puromycin (1 μg/ml), and pen/strep. HIV-1 NL4-3 was propagated as described previously (Adachi et al., 1986; van Marle et al., 2005). Z-FA-FMK was purchased from Imgenex (San Diego, Calif.).

### Immunoblot, Immunostaining, and Facs Analysis

Cell lysates were prepared by sonication in a buffer containing 10 mM Tris (pH 7.4), 2 mM EDTA, 1% NP-40, 50 mM mercaptoethanol, 100 μg/ml leupeptin and 2 μg/ml aprotinin. The lysates were then cleared by centrifugation at 16,000 g for 15 min, normalized for protein amount, mixed with SDS sample buffer, boiled for 5 min and stored at −70° C. After separation by SDS-PAGE, proteins were transferred to nitrocellulose membranes and detected by immunoblot hybridization. The primary antibodies (Abs) were as follows: anti-reovirus polyclonal Ab (Strong et al., 1998), anti-caspase 3 Ab (Imgenex, San Diego, Calif.), anti-PARP Ab (BD Biosciences, San Jose, Calif.), anti-XIAP Ab (Imgenex), and anti-actin Ab (Cell Signaling, Beverly, Mass.). The secondary Abs were horseradish peroxidase-conjugated anti-mouse Ab or horseradish peroxidase-conjugated anti-rabbit Ab (Pierce Biotech, Rockford, Ill.). FITC conjugated influenza type A antiserum was purchased from DakoCytomation Ltd (Cambridgeshire, United Kingdom). For immunostaining analysis, HIV infected cells were fixed/permeabilzed with cytofix/cytoperm (BD Bioscience, San Jose, Calif.), then incubated with HIV antiserum (cat. #:ab20460, abeam, Cambridge, Mass.) and secondary FITC antibody (Cedarlane, Ontario, Canada). For FACS analysis, cells were fixed/permeabilzed with cytofix/cytoperm (BD Bioscience, San Jose, Calif.), then incubated with reovirus antiserum (Kim et al., Oncogene) and secondary FITC antibody (Cedarlane, Ontario, Canada) for flow cytometry analysis.

### Electron Microscopy

EM preparation was performed by the University of Calgary Microscopy & Imaging Facility. Cells grown on glass slides were rinsed briefly with PBS (phosphate buffered saline) and fixed with 4% EM grade glutaraldehyde in 0.1 M cacodylate buffer (pH 7.4) for 1 hr at room temperature (RT). After washing three times with the same buffer, cells were post-fixed in 1% osmium tetroxide buffered with 0.1 M cacodylate for 1 hr at RT. Then cells were rinsed twice briefly with distilled water and stained en bloc for 30 min in 0.5% aqueous uranyl acetate. The cells were then dehydrated in an ethanol series and embedded in Spurr's low viscosity resin. Thin sections were cut with a diamond knife on a Reichert Ultracut E, stained with uranyl acetate and lead citrate and examined with a Hitach H-7000 transmission electron microscope at 75 kV.

### Animal Studies

SCID mice (6-8 weeks, Charles River, Wilmington, Mass.) received a single subcutaneous implantation of 5×10⁶ cells of HT1080 cells suspended in PBS. After palpable tumors were established (11 days after implantation), tumors were injected with Live reovirus (L Reo; n=5 tumors), UV-inactivated reovirus (D Reo; n=5 tumors), Z-FA-FMK (FA; n=5 tumors) and Live reovius suspended in Z-FA-FMK solution (Reo+FA; n=5 tumors). Tumor growth was measured externally using calipers and the volume was determined by the equation V=(L×W²)×0.5, where L is the largest dimension and W is the largest dimension perpendicular to L. Z-FA-FMK was dissolved in DMSO and administered as 0.02 mg suspended in PBS intratumorally per mouse every day up to 7 days post viral injection, and every 2 days until completion of the experiment. All the mice were treated according to protocols approved by the University of Calgary Animal Care Committee.

### Immunohistochemistry

All specimens taken from animals were fixed in 10% buffered formalin solution at room temperature. For reovirus antigen detection (reovirus structural proteins), deparaffinized tumor sections were retrieved in a solution containing 50 mM Tris (pH 7.5), 120 mM NaCl, 0.2% Tween 20, and 0.1% Triton X-100. After blocking the sections with a solution containing 50 mM Tris (pH 7.5), 120 mM NaCl, 0.2% Tween 20, 0.1% Triton X-100 and 2% normal goat serum for 1 hr, the sections were immunostained with a solution containing 0.1% reovirus antiserum, 50 mM Tris (pH 7.5), 120 mM NaCl, 0.2% Tween 20, 0.1% Triton X-100 and 2% normal goat serum for 2 hr. As a secondary antibody, biotinylated goat anti-mouse antibody (Vector Laboratories) was used at 1:100 in a solution containing 50 mM Tris (pH 7.5), 120 mM NaCl, 0.2% Tween 20, 0.1% Triton X-100 and 2% normal goat serum for 2 hr at room temperature. Detection was monitored by a diaminobenzidine tetrahydrochloride-based immunohistochemistry protocol (DAB substrate kit) according to the suggestions of the manufacturer (Vector Laboratories, Burlingame, Calif.). Tumor slides were stained with the nickel solution from the kit. Dehydration was carried out in a series of graded ethanol solutions, followed by clarification in xylene. Slides were mounted with Vectamount (Vector Laboratories, Burlingame, Calif.) and stored at 25° C.

### Example 1

### PFMKs such as Z-FA-FMK Blocks Reoviral Replication and Cures Cells of a Persistent Infection with Reovirus In Vitro

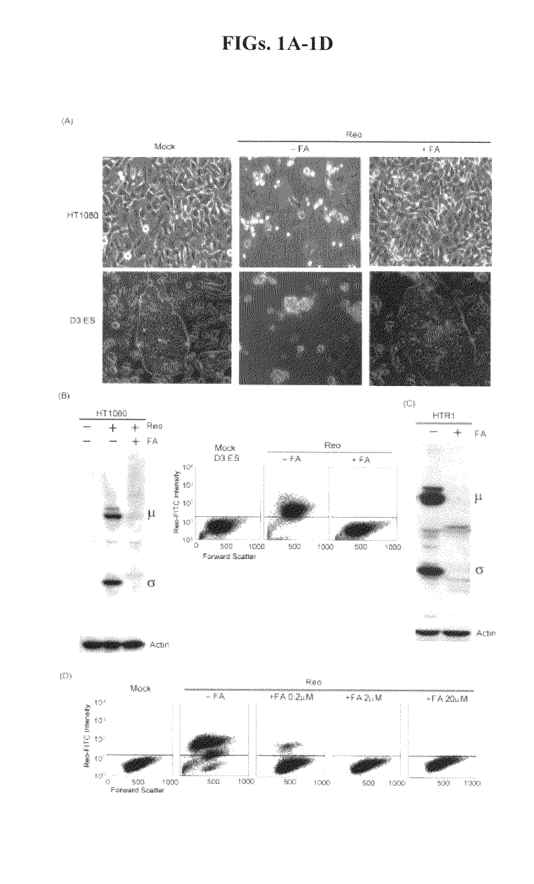

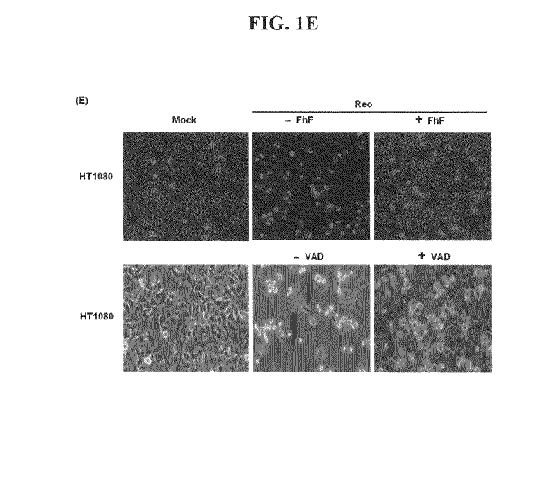

N-Benzyloxycarbonyl-Phe-Ala-fluoromethylketone (Z-FA-FMK) is a cell permeable cathepsin B inhibitor (Ahmed et al., 1992; Rasnick, 1985; Schotte et al., 2001). Since suppression of cellular cathepsin B partially affects reovirus replication by inefficient proteolytic disassembly of viral outer-capsid proteins (Ebert et al., 2004; Ebert et al., 2002), pharmacological inhibition of cellular cathepsin B activity by Z-FA-FMK may affect reovirus replication. Thus a reovirus susceptible cancer cell line, HT1080 (Kim et al., Oncogene), and a mouse embryonic stem cell line were treated with Z-FA-FMK. Unexpectedly, instead of a partial viral inhibition, Z-FA-FMK completely blocked reovirus replication (FIGS. 1A and 1B). Furthermore, Z-FA-FMK treatment cured persistently infected cells with reovirus (HTR1, 14) (FIG. 1C). Anti-viral activity of Z-FA-FMK was also evaluated by fluorescence-activated cell sorting (FACS) analysis. As shown in FIG. 1D, Z-FA-FMK effectively suppressed reoviral replication potential in a dose-dependent manner. Thus, Z-FA-FMK is a potent anti-reoviral inhibitor in vitro. In addition, other peptidyl fluoromethylketones (PFMKs) such as Mu-FhF-FMK (N-Morpholineurea-phenylalanyl-homophenylalanyl-fluoromethylketone) and Z-VAD-FMK (Z-Val-Ala-Asp-fluoromethylketone) effectively inhibited reovirus-mediated cytopathogenicity as shown in FIG. 1E.

### Example 2

### Z-FA-FMK Induces Defects in Reoviral Maturation

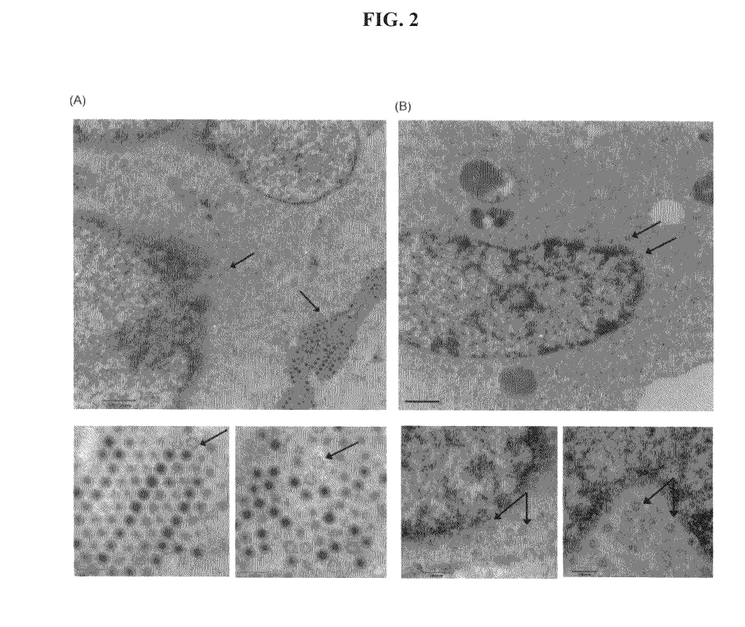

Although cellular cathepsin B activity plays a role in reoviral replication, it has been shown that cathepsin B inhibition alone is not sufficient to block reoviral replication (Ebert et al., 2004). Thus, Z-FA-FMK could significantly affect other steps of the reoviral infection cycle in addition to cathepsin B-mediated inhibition in order to exert the potent antiviral activity as shown above. Thus, Z-FA-FMK treated cells were treated under electron microscopy (EM) to examine virion morphologic changes after Z-FA-FMK treatment. As shown in FIG. 2, untreated cells developed normal appearing viral factories at 48 hr post-infection. However, Z-FA-FMK treated cells did not form these viral factories and scattered viral particles were detected with perinuclear localization. More interestingly, all of the scattered viral particles contained empty capsids (FIG. 2B) in comparison to crystalline arrays of full or empty capsids seen in the untreated cells (FIG. 2A) (Fields et al., 1971). Therefore, Z-FA-FMK affects a virion morphogenesis step resulting in inhibition of viral factory development.

Mammalian orthoreoviruses are believed to replicate in distinctive, cytoplasmic inclusion bodies, commonly called viral factories following virion entry, proteolytic activation and cytoplasmic transcription and translation. As shown in the EM analysis, there is a significant blockage of the reovirus virion packaging and subsequent viral factory development upon Z-FA-FMK treatment, resulting in an empty virus particle. Thus, Z-FA-FMK may also affect unknown viral/host factors involved in virion packaging and factory development during reovirus infection. Because the maturation processes, including packaging, of reovirus virion are unique, it is likely that Z-FA-FMK specifically affects a reovirus packaging step. It has been suggested that muNS, sigmaNS and sigma 3 play a role in the reovirus RNA packaging process (Nibert et al., 1996). Thus, it is possible that Z-FA-FMK may specifically affect the normal function of these packaging-related reoviral proteins.

### Example 3

### Z-FA-FMK Does not Affect Replication of Influenza a Virus or Hiv-1

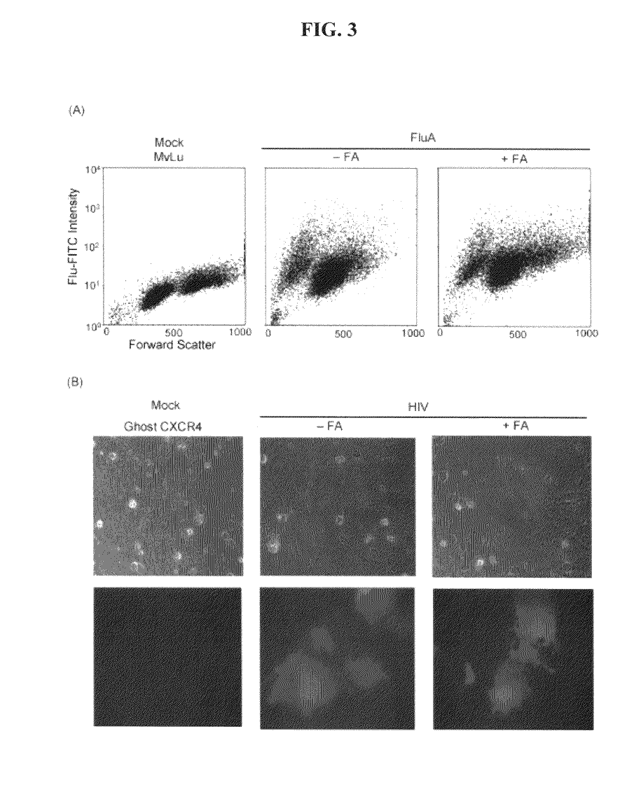

The effects of Z-FA-FMK on the following two viruses were studied: influenza A virus, which is known to utilize a cellular endocytic pathway during its infection cycle (Rust et al., 2004), and HIV type 1 virus by FACS and immunostaining analysis. However, Z-FA-FMK did not affect viral replication activity of influenza A and HIV-1 (FIG. 3). In comparison to reovirus and myxoma viruses, it is likely that influenza and HIV viruses are less dependent on cellular protease activities for virus entry and maturation. Thus Z-FA-FMK may have decreased inhibitory effects on these viruses.

### Example 4

### Z-FA-FMK Blocks Reovirus Infection In Vivo

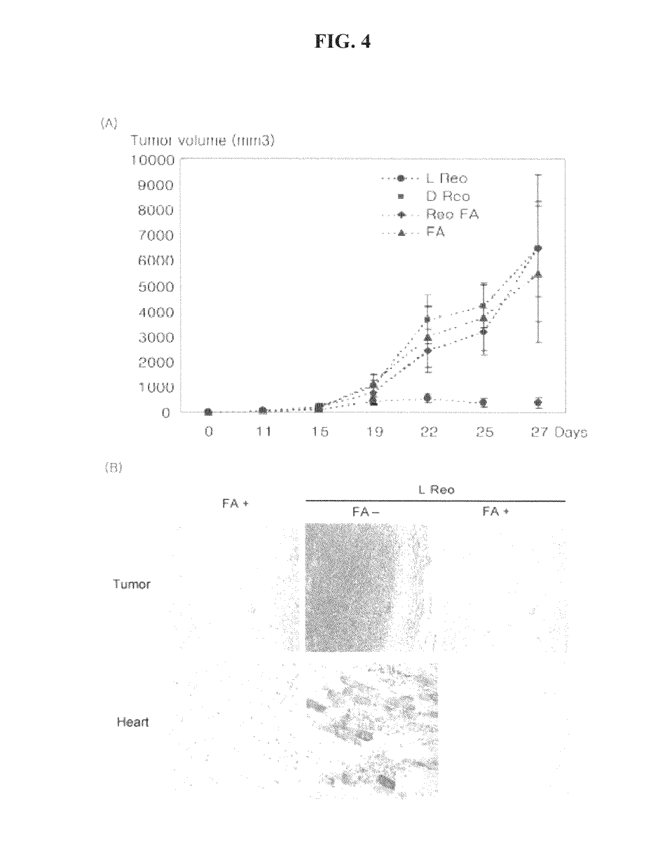

Because inhibition of reoviral replication by Z-FA-FMK is so effective in vitro, it may also affect reoviral infection in vivo. To evaluate in vivo antiviral activity of Z-FA-FMK, a tumor xenograft model using SCID mice was used since reovirus replicates well in Ras-oncogenic tumors in vivo (Coffey et al., 1998; Kim et al., 2007; Strong et al., 1998). Because SCID mice are highly susceptible to reovirus-induced viral myocarditis (Kim et al., 2007; Loken et al., 2004), viral replication activity of both tumor and host heart can be monitored by this tumor xenograft model. Thus Ras-oncogenic HT1080 cells were xenografted in SCID mice and either reovirus alone or reovirus supplemented with Z-FA-FMK was administrated intratumorally. At 16 days post-viral injection, tumor and heart tissues were examined by immunohistochemical analysis. As shown in FIGS. 4A-4B, reovirus treated mice showed extensive viral replication at both tumor sites and heart tissues. Viral replication of the tumors caused suppression of tumor growth and viral replication of the host heart tissues induced massive lymphocyte infiltration, which is a typical pathologic manifestation of viral myocarditis (FIG. 4B). However, reovirus replication activity of both tumor and heart tissues was effectively blocked by Z-FA-FMK treatment (FIG. 4B). Therefore, Z-FA-FMK systemically and effectively inhibits reovirus infection in vivo.

### Example 5

### Cathepsin B Inhibitor Assay

As mentioned, various methods exist in the art for determining whether a compound is a cathepsin B inhibitor. See, e.g., U.S. Pat. No. 5,068,354; Barrett and Kirschke, 1981. The following protocol describes one such method.

To each of 0.95 ml of reaction solutions containing 2.5 mM 2-mercaptoethanol, 1 mM disodium ethylenediaminetetraacetate, 0.1 M sodium potassium phosphate buffer (pH 6.0), 0.1% brij-35 (Nacalai Tesque Inc.), 1% dimethyl sulfoxide and different concentrations of the compound of interest is added 25 μl of 200 nM cathepsin B solution (Sigma Chemical Co.), and the mixture is preincubated at 40° C. for 10 minutes, after which 25 μl of 200 M benzyloxycarbonyl L-phenylaranyl-L-arginine 4-methylcoumaryl-7-amide (Peptide Institute Inc.) is added for starting the reaction. After incubation at 40° C. for 10 minutes, the reaction is stopped by addition of 1 ml of 100 mM sodium chloroacetate in 100 mM sodium acetate (pH 4.3). The fluorescence of the liberated 7-amino-4-methylcoumarine is determined using a Shimazu fluorimeter RP-5000 with excitation at 380 nm and emission measured at 440 nm. Determination of the reduction of cathepsin B activity can be determined by comparing the inhibition rate with the rate measured in a similar manner to the above but without the test drug.

### Example 6

### Z-FA-FMK Inhibitory Effect on a Leporipoxvirus (Myxoma Virus)

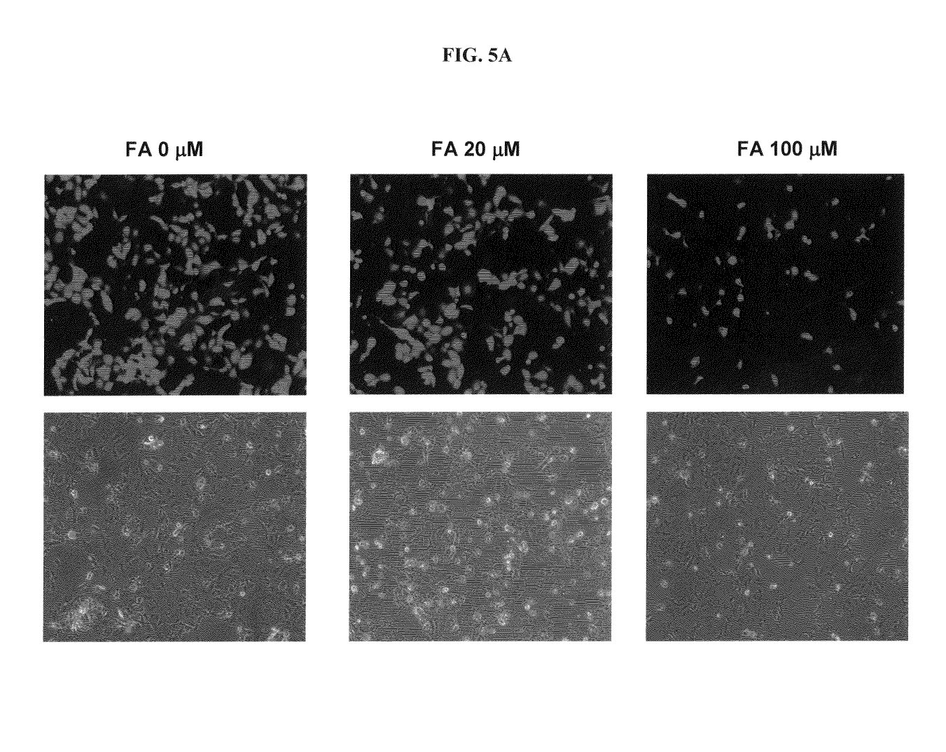

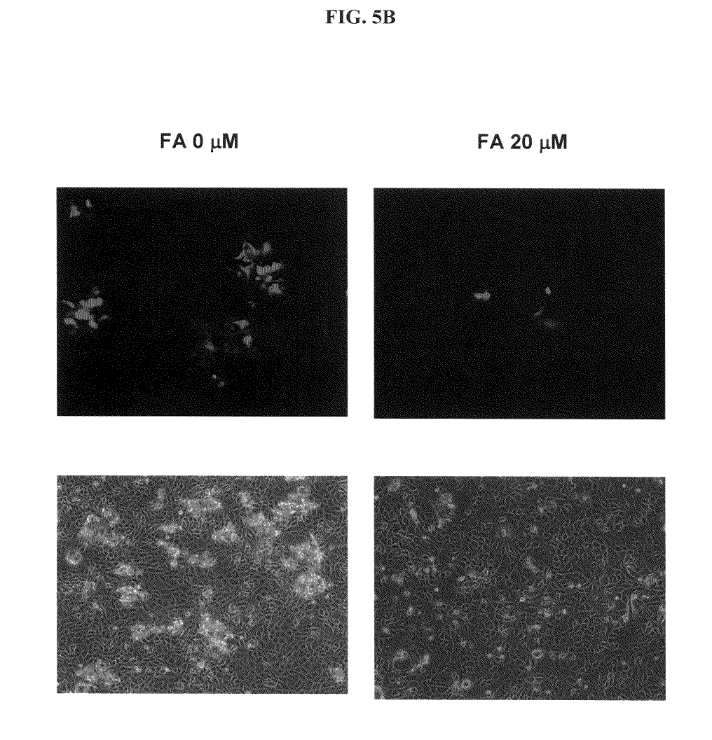

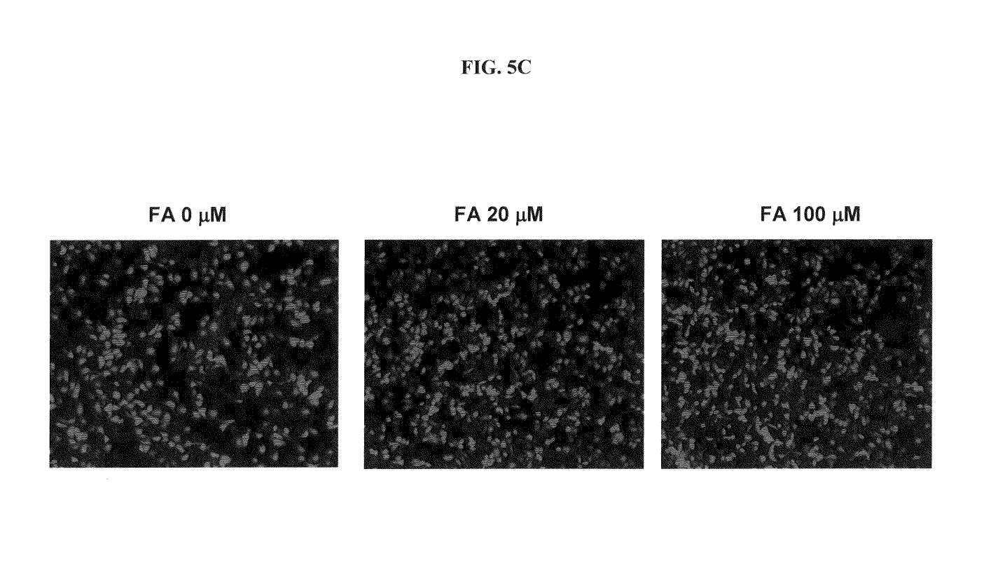

The effects of Z-FA-FMK were also studied using a leporipoxvirus such as myxoma virus. Because poxvirus utilizes various cellular machineries including various cellular proteases for viral propagation, myxoma virus was examined to see whether Z-FA-FMK can affect virus propagation. Since myxoma virus propagates very well in BGMK (buffalo green monkey kidney) cell monolayer, we used BGMK cells infected with myxoma virus. As shown in FIG. 5A-B, Z-FA-FMK effectively suppressed myxoma virus propagation (foci development). Unlike reovirus, myxoma virus propagation was mostly dependent on direct cell to cell spread (few extracellular viral particles during infection cycle). Thus, inhibition of foci development is indicative of the inhibition of viral propagation. When BGMK cells were infected with Myx-GFP at MOI of 0.5 in the presence of Z-FA-FMK, at 2 days post-infection, GFP expression level of BGMK monolayers were significantly suppressed and myxoma virus foci development was significantly suppressed as Z-FA-FMK dosages were increased. (FIG. 5A). When BGMK cells were infected with Myx-GFP at very low MOI (MOI of 0.01) in the presence of Z-FA-FMK, the inhibition of foci development is more readily detectable (FIG. 5B). However, when BGMK cells were infected with Myx-GFP at high MOI (MOI of 3) in the presence of Z-FA-FMK, the Z-FA-FMK inhibitory effects were not detectable as shown by widespread GFP expressing cells (FIG. 5C), suggesting that Z-FA-FMK may not directly block an initial virus propagation stage.

All of the methods disclosed and claimed herein can be made and executed without undue experimentation in light of the present disclosure. While the methods of this invention have been described in terms of preferred embodiments, it will be apparent to those of skill in the art that variations may be applied to the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### 5. REFERENCES

The following references, to the extent that they provide exemplary procedural or other details supplementary to those set forth herein, are specifically incorporated herein by reference:
U.S. Pat. No. 5,068,354
U.S. Pat. No. 5,210,272
U.S. Pat. No. 5,344,939
U.S. Pat. No. 5,698,519
U.S. Pat. No. 6,124,257
U.S. Pat. No. 6,534,498
U.S. Pat. No. 6,596,715
U.S. Pat. No. 6,605,589
U.S. Pat. No. 6,162,828
U.S. Pat. No. 7,151,163
U.S. Pat. No. 7,163,678
U.S. Prov. Appln. 60/704,604
U.S. Prov. Appln. 60/906,706
Ahmed et al., Biochem. Pharmacol., 44:1201-1207, 1992.
Barrett and Kirschke, Methods Enzymol., 800:535-561, 1981.
Baty and Sherry, J. Virol., 67:6295-6298, 1993.
Bodanszky et al., In: The Practice of Peptide Synthesis, Springer-Verlag, NY, 1984.
Bodanszky, In: Peptide Chemistry: A Practical Textbook, Springer-Verlag, NY, 1988.
Chandran and Nibert, J. Virol., 72(1):467-475, 1998.
Coffey et al., Science, 282:1332-1334, 1998.
Dilakian and Tsvetkova, Biomed Khim. 51(5):485-500, 2005.
Duncan and Stanish, J. Virol., 28:444-449, 1978.
Duursma and Agami, Semin. Cancer Biol., 13:267-273, 2003.
Ebert et al., J. Biol. Chem., 277:24609-24617, 2002.
Ebert et al., J. Biol. Chem., 279:3837-3851, 2004.
Esser et al., J. Rheumatol. 20:1176-1183 (1993).
Evans et al., J. Amer. Chem. Soc., 112:4011-4030, 1990.
Fields et al., Virology, 43:569-578, 1971.
Fields, In: Retroviridae: The viruses and their replication,” 3^{rd} Ed., Lippincott-Raven Publ, Phila., 58:1553-1771, 1996.
Gotoh et al., Appl. Microbiol. Biotech., 56:742-749, 2001.
Greene and Wuts, Protecting Groups in Organic Synthesis, 3^{rd} ed., John Wiley & Sons, Inc., 1999.
Gross et al., In: The Peptides. Analysis, Synthesis, Biology, Vols. 1, 2, 3, 5 and 9, Academic Press, NY, 1980-1987.
Halvorsrud and Orstavik, Scand. J. Infect. Dis., 12(3):161-164, 1980.
Harrison et al., Proc Natl Acad Sci USA, 101: 11178-11192, 2004.
Hashiro et al., Arch. Virol., 54:307-3015, 1977.
Heggie and Gaddis, Pediatr. Res., 13:937-941, 1979.
Hirasawa et al., Cancer Res., 63:348-353, 2003.
Holzel et al., J. Infect., 2(1):33-37, 1980.
Institute of Medicine (1999) Assessment of Future Scientific Needs for Variola Virus, ed.

Briere, R. (Natl. Acad. Press, Washington, D.C.).
Kapikian et al., J. Infect. Dis., 174(1):S65-72, 1996.
Kilakian and Tsvetkova, Biomed Khim., 51:485-500 (2005).
Kim et al., Oncogene (2007) (in press).
Lane et al., Nat. Med. 7:1271-1273 (2001).
Lipton, Physiol. Rev., 79:1431-1568 (1999).
Loken et al., Cancer Biol. Ther., 3:734-738, 2004.
McFadden, Nat. Rev. Microbiol., 3:201-213, 2005.
Moss, B., In: Fields Virology, eds. Fields et al. (Eds.), Lippincott Williams & Wilkins, Philadelphia, pp. 2849-2883, 2001.
Nibert et al., In: Fields Virology, Fields et al. (Eds.), 3^{rd} Ed., Lippincott-Raven, PA, 1557-1596, 1996.
Nomura and Katunuma, J. Med. Invest., 52:1-9, 2005.
Norman and Lee, Drug Disc. Today, 10:847-855, 2005.
PCT/US2006/029881
Pham, Immun. 679-691, 2005.
Priscott, Br. J. Exp. Pathol., 64:467-473, 1983.
Pu et al., J. Amer. Chem. Soc., 56:1280-1283, 1991.
Rasnick, Anal. Biochem., 149:461-465, 1985.
Remington's Pharmaceutical Sciences, 18th Ed. Mack Printing Company, 1990.
Rust et al., Nat. Struct. Mol., Biol., 11:567-573, 2004.
Sabin, Science, 130:1387-1389, 1959.
Schotte et al., J. Biol. Chem., 276:21153-21157, 2001.
Senkevich, et al., J. Virol. 78:2357-2366, 2004.
Saville et al., J. Gen. Virol., 83:685-694, 2002.
Smith et al., Virology, 39:791-810, 1969.
Smith et al., J. Gen. Virol. 83:2915-2931, 2002.
Stewart and Young, In: Solid Phase Peptide Synthesis, 2^{nd} Ed., Pierce Chemical Co., Rockford, Ill., 1984.
Strong et al., EMBO J, 17:3351-3362, 1998.
Takahashi et al., BioFactors, 10:339-345, 1999.
Terheggen et al., Eur. J. Clin. Microbiol. Infect Dis., 22:197-198, 2003.
Tyler and Fields, In: Fields Virology, Fields et al. (Eds.), 3^{rd} Ed., Lippincott-Raven, PA, 1597-1623, 1996.
Vasiljeva et al., Curr. Pharm. Des., 13:385-401, 2007.
Vodros and Fenyo, Methods Mol. Biol., 304:333-342, 2005.
Weiner et al., Proc. Natl. Acad. Sci. USA, 74:5744-5748, 1977.
Williams et al., J. Amer. Chem. Soc., 113:9276-9286, 1991.
Williams, In: Synthesis of Optically Active alpha.-Amino Acids, Pergamon Press, 1989.

What is claimed is:

1. A method of inhibiting viral replication in a cell, comprising contacting the cell with an effective amount of a peptidyl fluoromethylketone (PFMK), wherein the virus is a Reoviridae virus or a Poxyiridae virus.

2. The method of claim 1, wherein the PFMK is a cathepsin inhibitor or a cysteine protease inhibitor.

3. The method of claim 2, wherein the cathepsin inhibitor is a cathepsin B inhibitor.

4. The method of claim 1, wherein the virus is a Reoviridae virus.

5. The method of claim 4, wherein the virus is orthoreovirus or rotavirus.

6. The method of claim 5, wherein the virus is a reovirus.

7. The method of claim 6, wherein the reovirus is a wild-type reovirus or an attenuated reovirus.

8. The method of claim 7, wherein the virus is an attenuated reovirus.

9. The method of claim 8, wherein the attenuated reovirus lacks a wild-type reovirus S1 gene.

10. The method of claim 1, wherein the virus is a Poxyiridae virus.

11. The method of claim 10, wherein the Poxyiridae virus is a chordopoxyirinae virus.

12. The method of claim 11, wherein the chordopoxyirinae virus is selected from the group consisting of orthopoxvirus and leporipoxvirus.

13. The method of claim 12, wherein the chordopoxyirinae virus is an orthopoxvirus.

14. The method of claim 13, wherein the orthopoxvirus is selected from the group consisting of vaccinia virus, monkeypox virus, cowpox and variola virus.

15. The method of claim 12, wherein the chordopoxyirinae virus is a leporipoxvirus.

16. The method of claim 15, wherein the leporipoxvirus is myxoma virus.

17. The method of claim 1, wherein the PFMK is Z-FA-FMK.

18. The method of claim 1, wherein viral replication is inhibited by at least about 50%.

19. The method of claim 1, wherein viral replication is inhibited by at least about 75%.

20. The method of claim 1, wherein viral replication is inhibited by at least about 99%.

21. The method of claim 1, wherein viral replication is inhibited by about 100%.

22. The method of claim 1, wherein the cell is comprised in neural tissue.

23. The method of claim 1, wherein the cell is comprised in cardiac muscle tissue.

24. The method of claim 1, wherein the cell is a cancer cell.

25. The method of claim 22, wherein the cancer cell is a lung cancer cell, a colon cancer cell, a pancreatic cell, a thyroid cell, a white blood cell, or a melanocyte.

26. The method of claim 24, wherein the cancer cell is comprised in a carcinoma.

27. The method of claim 26, wherein the carcinoma is selected from the group consisting of melanoma, adenocarcinoma, squamous cell carcinoma, small cell carcinoma and oat cell carcinoma.

28. The method of claim 24, wherein the cancer cell is comprised in a sarcoma.

29. The method of claim 28, wherein the sarcoma is selected from the group consisting of fibrosarcoma, chondrosarcoma, osteosarcoma, hepatoma and neuroblastoma.

30. The method of claim 24, wherein the cancer cell is comprised in a hematopoietic malignancy.

31. The method of claim 30, wherein the hematopoietic malignancy is selected from the group consisting of lymphoma, leukemia, myeloma, myelodysplastic syndrome and myeloproliferative disorders.

32. The method of claim 1, wherein the cell is persistently infected with a virus.

33. The method of claim 32, wherein the virus is a reovirus.

34. The method of claim 1, wherein the method takes place in vitro.

35. The method of claim 34, wherein the cell is comprised in a mixed cell population.

36. The method of claim 35, wherein the mixed cell population comprises stem cells.

37. The method of claim 36, wherein the stem cells comprise hematopoietic stem cells.

38. The method of claim 1, wherein the method takes place in vivo.

39. The method of claim 1, wherein the cell is comprised in a subject.

40. The method of claim 39, wherein the subject is a mammal.

41. The method of claim 40, wherein the mammal is a human.

42. The method of claim 41, wherein the human is less than one year old.

43. The method of claim 41, wherein the human is a pregnant human.

44. The method of claim 41, wherein the human is infected with a virus.

45. The method of claim 44, wherein the virus is a reovirus.

46. The method of claim 39, wherein the subject has cancer.

47. The method of claim 46, wherein the subject is undergoing anti-cancer therapy.

48. The method of claim 47, wherein the anti-cancer therapy is reoviral anti-cancer therapy.

49. The method of claim 39, wherein the subject is immunocompromised.

50. The method of claim 1, wherein the PFMK is comprised in a pharmaceutically acceptable composition.

51. A method of inhibiting viral replication in a subject, comprising administering an effective amount of a PFMK to the subject.

52. The method of claim 51, wherein the virus is a Reoviridae virus or a Poxyiridae virus.

53. The method of claim 51, wherein PFMK is administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, subcutaneously, subconjunctival, intravesicularlly, mucosally, intrapericardially, intraumbilically, intraocularally, orally, locally, via inhalation, via injection, via infusion, via continuous infusion, via localized perfusion bathing target cells directly, via a catheter, via a lavage, in cremes, in lipid compositions, or any combination thereof.
